(19) United States
(12) Patent Application Publication
(10) Pub. No.: US 2019/0126054 A1
(43) Pub. Date: May 2, 2019

(54) DEVICE FOR HIGH-VOLTAGE THERAPY

(71) Applicant: BIOTRONIK SE & CO. KG, BERLIN (DE)

(72) Inventors: INGO WEISS, BERLIN (DE); MICHAEL FRIEDRICH, KLEINMACHNOW (DE); THOMAS DOERR, BERLIN (DE); ULRICH FEESE, BERLIN (DE); KARSTEN SCHLODDER, FUERSTENWALDE (DE); GERNOT KOLBERG, BERLIN (DE)

(21) Appl. No.: 16/171,613

(22) Filed: Oct. 26, 2018

(30) Foreign Application Priority Data
Oct. 26, 2017 (DE) 10 2017 125 044.1

(51) Int. Cl. A61N 1/39; A61N 1/375

(52) U.S. Cl. CPC A61N 1/3968; A61N 1/37512; A61N 1/3975; A61N 1/3956

(57) ABSTRACT

A configuration performs electrical therapy on a patient and is configured to produce at least one electrical therapeutic voltage, which acts at a therapy site in or on the patient's body. The configuration contains at least one first and one second subunit, and every subunit has at least one energy storage. The amount of energy storable by every individual energy storage is smaller than the amount of energy required to produce the electrical voltage for therapy.

### CROSS-REFERENCE TO RELATED APPLICATION

This application claims the priority, under 35 U.S.C. § 119, of German application DE 10 2017 125 004.1, filed Oct. 26, 2017; the prior application is herewith incorporated by reference in its entirety.

### BACKGROUND OF THE INVENTION

### Field of the Invention

The subject of the invention is devices and systems for electrical stimulation therapy on a patient. In particular, the invention relates to the area of cardiac stimulation; in a specific example, it relates to implantable systems for cardioversion and defibrillation of the heart.

Implantable systems are known for cardiac stimulation and for cardioversion and defibrillation (implantable cardioverter-defibrillators, ICDs) that consist of an implant housing and that comprise energy supply, capacitors, electronics modules, etc., and one or more electrode leads. The electrode leads have one or more electrodes for measuring cardiac potentials and/or outputting stimulation pulses. Electrode leads with a shock electrode (shock coil) are able to output an intracardiac defibrillation shock in the case of life-threatening cardiac arrhythmias. The electrode leads are connected with the implant housing through connector connections integrated in a header module.

The prior art also discloses cardiac stimulation and cardioversion and defibrillation electrotherapy systems in which the cardioversion and shock function is offloaded to a subcutaneous implant, a so-called subcutaneous implantable cardioverter-defibrillator (sICD), which performs extravascular defibrillation. The sICD is implanted under the patient's skin, at least two electrode poles for outputting the shock being arranged in such a way that the current path (“shock vector”) leads through the areas of the heart that are to be shocked. sICDs are known that consist of an implant housing and a connected shock electrode lead, the housing being implanted on the side over the ribs near the axilla, and the shock electrode lead being implanted in the middle, over the chest.

The advantages of a sICD system over an ICD system are that no intracardiac electrode has to be placed, reducing the risk for the patient. The disadvantage of a sICD system is the higher shock energy that is required due to the longer current path from the shock electrodes to the cardiac tissue. For instance, known sICD systems require high therapeutic voltages of over 1,400 volts and energies of over 80 joules per shock. Thus, the hardware requirements on such sICD systems are correspondingly higher than those of conventional ICDs. Special expensive high-voltage circuit elements (charging circuit, high-voltage capacitors) and spacious layouts are required to comply with the breakdown paths, etc. The more specialized hardware components increase the volume of the implant housing of the sICD, so that the device is clearly larger than conventional ICDs, and thus bothers the patient substantially more.

### SUMMARY OF THE INVENTION

Therefore, it is the goal of this invention to develop a therapy system that does not have the above-mentioned disadvantages and is lighter and can be implemented more economically than known solutions. The invention is intended to solve the problem that electrotherapy devices (implantable or non-implantable) often require a large-volume design and the use of expensive special circuit elements to produce the energies and voltages required for therapy. In particular, the inventive solution is intended to provide an effective therapeutic voltage for extravascular defibrillation, without this requiring special, large-volume high-voltage circuit elements.

The invention accomplishes this goal by the features of the independent claims. Favorable embodiments and advantages of the invention follow from the other claims and the description.

A first aspect of the invention describes an arrangement for electrical therapy on a patient, the arrangement being designed to produce at least one electrical therapeutic voltage, which acts at a therapy site in or on the patient's body. The arrangement comprises at least one first and one second subunit, and every subunit has at least one energy storage. The amount of energy storable by every individual energy storage is smaller than the amount of energy required to produce the electrical voltage for therapy.

The subject of the invention is an arrangement for electrical therapy on a patient, the arrangement having at least two subunits. Each subunit makes available a voltage for electrical therapy, the voltage provided by every a subunit being smaller than the therapeutically effective voltage achieved at a therapy site.

Preferably, according to one embodiment of this invention every subunit has at least one of the following components:

A detection unit to detect body parameters;
A therapy unit to produce and/or output a voltage for electrical therapy;
A controller; and/or
A communication unit.

According to one aspect of the inventive solution, the required therapeutic voltage is produced by superimposing partial contributions from multiple subunits of the arrangement.

According to a preferred embodiment of the inventive arrangement, each of the at least first and second subunits is designed to produce a voltage by use of energy from the energy storage. The arrangement is configured to produce the electrical therapy voltage by superimposing the voltages produced by the at least first and second subunits.

In one embodiment of this invention, the voltages produced by the at least two subunits are superimposed so that the amplitude of the superimposed voltage is higher than the amplitude of a voltage produced by one subunit.

In one embodiment of this invention, the first subunit is arranged in a first outer housing and the second subunit is arranged in a second outer housing. For example, the at least first and second subunits are individual components of a therapy system, such as a battery and capacitor. If these large-volume components are put in separate housings and connected together, the device volume of such a therapy device is distributed between two individual devices. The at least two subunits can also be individual therapy devices, such as, for example subcutaneous implantable cardioverter-defibrillators (sICDs), or one therapy device and one device for measuring physiological signals without a therapeutic function, such as, e.g., in the area of heart implants a loop recorder for long-term recording of cardiac signals.

According to another embodiment of this invention, the at least first and second subunits are arranged in a common outer housing. For example, it is conceivable for two therapy devices that can produce a voltage for electrical stimulation to be arranged in one housing. To produce a therapeutic voltage, the respective voltages are output so that at the therapy site they are superimposed constructively/essentially phase-synchronized, giving the therapeutic voltage a high amplitude. The volume of every individual therapy device can be smaller than if a single therapy device would be developed, reducing the total volume and providing freedom in the design of the hardware architecture and arrangement.

According to a preferred embodiment of this invention, the voltages produced by the at least two subunits are superimposed so that the voltages are essentially phase-synchronized at the therapy site. In the context of the invention, essentially phase-synchronized should be understood to mean that the voltages are electrical alternating voltages that are superimposed at the therapy site in such a way that the amplitude of the superimposed voltage (which represents the electrical therapy voltage at the therapy site) is higher than the amplitude of every one of the individual alternating voltages. Preferably, the alternating voltages have the same frequencies and a phase shift of 0°+/−2π. Small tolerances in phase shift and frequency are unavoidable and are included.

According to one embodiment of this invention, every subunit is configured to communicate with at least one other subunit, the communication being galvanic, electromagnetic, optical, mechanical, and/or acoustic. To accomplish this, the subunit can have a corresponding transmitter/receiver.

In a preferred embodiment of the invention, at least one subunit has a controller that is configured to issue a therapy command carrying information about at least one of the following parameters:

a) Information about the phase of the voltage provided by the respective subunit;
b) The point in time that the therapy is output;
c) The polarity of the therapy that is output; and/or
The duration of the therapy that is output from the respective subunit.

In one embodiment of the invention, one subunit assumes a master role with respect to producing and/or outputting a voltage. The at least one other subunit assumes a slave role, so that the at least one subunit playing a slave role orients itself about the point in time and/or polarity and/or duration of the therapy output on the basis of the subunit playing the master role.

Preferably, the subunit that assumes the master role is the one that first receives a body parameter calling for therapy, i.e., the subunit that detects it before all other subunits.

In one example, all subunits detect a physiological signal. As soon as a first subunit measures a physiological event that requires therapy, the subunit produces a voltage and outputs the voltage at the therapy site. As soon as the at least one other subunit detects the voltage output of the first subunit, this subunit also begins producing and outputting the voltage.

According to another aspect of the invention, the arrangement has at least one connection arrangement that electrically connects the at least two subunits and that is electrically insulated from the patient's body. The at least two subunits can have a series, anti-series, parallel, or star-shaped connection through the connection arrangement.

According to one embodiment of the invention, the voltages produced by at least two subunits are essentially phase-synchronized at the therapy site by the arrangement through the at least one connection arrangement.

In other inventive embodiments of the invention, the at least two subunits comprise:

a) one battery each; and/or
b) one capacitor each; and/or
c) each of the subunits is a subcutaneously implantable cardioverter-defibrillator (sICD).

According to the invention, the spatial separation of the different components required for producing a therapeutic voltage into different subunits makes it possible to dispense with a single large-volume device. The invention also comprises the idea of having individual smaller-volume therapy devices work together to produce a high therapeutic voltage. Each of these smaller therapy devices can have its own outer housing, or they can be arranged in a common outer housing. The invention provides configuring multiple devices so that they can, in coordinated operation, produce a therapeutic voltage at the therapy site. The therapeutic voltage can be produced by superimposing or adding, at the therapy site, the voltages that are produced by individual subunits, so that the required therapeutic energy/therapeutic voltage can be applied.

In a preferred embodiment of the inventive arrangement, at least one subunit is designed to record an electrocardiogram or a subcutaneous electrocardiogram.

In an advantageous embodiment of the inventive arrangement, the subunits are sICDs. Two or more sICDs are interconnected so that the voltages are added together at the therapy site. In one embodiment of the inventive arrangement, the subunits are connected in anti-series. According to one sample embodiment of the invention, each of the subunits belongs to a voltage class under 1,000 volts. The inventive arrangement makes it possible, by superimposing the voltages of the subunits, to produce a therapeutic voltage that lies above the voltage of one subunit (here over 1,000 volts, e.g., for extravenous defibrillation). This makes it possible, using economical hardware technology of the voltage class less than 1,000 V, to generate a therapeutic voltage that lies over 1,000 volts.

Another aspect of this invention involves phase-synchronized superimposition of the voltages of the subunits. This allows targeted generation of an effective therapeutic voltage. To ensure phase-synchronized superimposition of the voltages, the subunits that are involved are synchronized. In one embodiment of the inventive arrangement, the subunits are synchronized by galvanic, optical, electromagnetic, or mechanical/acoustic communication between the subunits.

The subject of the invention is a high-voltage therapy device that is implanted in the body in a distributed manner, this high-voltage therapy device being characterized in that the energy storage elements are put in locally separated subunits that are electrically connected with one another in such a way that the therapeutic effect is realized by synchronized superimposition of partial contributions from the individual subunits. The synchronization is accomplished by galvanic, electromagnetic, optical, mechanical, or acoustic communication between the subunits.

In one embodiment of the invention, the arrangement has at least one connection element through which the subunits are electrically connected. The connection element is electrically insulated from the patient's body.

In another embodiment, the subunits have at least one connection, in order to connect at least one other subunit through a connection cable.

Preferably, every subunit has at least one energy storage element. The energy storage elements are, for example, batteries (primary cells and/or secondary cells) or capacitors or a combination/interconnection of them.

In one embodiment, it would be conceivable for such an interconnection to have energy conversion means, such as, e.g., a charging circuit that converts low battery voltages into high therapeutic voltages, under the control of a controller. Additionally or alternatively, such an interconnection can have switches/therapy switches according to which the therapeutic voltage or the partial amount of the therapeutic voltage is additionally connected, under the control of the controller.

According to a preferred embodiment of this invention, the subunits are connected together and synchronized in such a way that the therapeutic current results by summation of the partial currents generated by the individual subunits. For addition of the currents (e.g., parallel connection of the subunits), the subunits are connected together through more than one connection conductor or the connection cable contains more than only one conductor.

In another embodiment of this invention, one of the subunits is realized or programmed/configured as a master, and the at least one other subunit is realized or programmed/configured as a slave. In this connection, “master role” means that if, in a measurement signal, a physiological event is detected that makes therapy necessary, the subunit in the master role takes over guidance by coordinating, communicating, synchronizing, and outputting therapy with the other subunits. Accordingly, the subunits in the slave role subordinate themselves to the subunit in the master role, i.e., in coordinating, communicating, synchronizing, and outputting therapy with the other subunits they orient themselves on the basis of the subunit in the master role.

Preferably, the subunit working as master has a detection unit (including a sensing unit) that derives the necessity of outputting therapy from analyzing intracorporeal signals.

An inventive embodiment provides that multiple subunits or all subunits record an electrical measurement signal representing physiological properties of the patient, for example an electrocardiogram (ECG). Furthermore, multiple subunits or all subunits are designed to detect events in the electrical measurement signal that require outputting a therapy. In one embodiment, the subunit that assumes the master role is the one that first makes a detection decision. Furthermore, the subunit that becomes master can put the other subunits into a slave role.

In one embodiment of the invention, each of the subunits comprises a controller to trigger the therapy command. Furthermore, each of the subunits has a communication unit, which serves to transmit and receive the therapy command, among other things (In this case, the communication information is the therapy command). Transmission and reception should be understood to include galvanic coupling.

For example, the subunit in the master role forwards a therapy command both to its own therapy unit and also to the slave subunits.

In a preferred embodiment, the therapy command is issued in the form of a signal that encodes the point in time at which the therapy is output, the polarity of the therapy that is output, and the duration for which the partial energies of the individual subunits are additionally connected. This ensures that the partial contributions from the individual subunits are superimposed synchronously and with the right sign.

In one sample embodiment of this invention, the subunits further comprise highly accurate time measurement devices, e.g., devices that are accurate to within 5 ms. If the subunit in the master role recognizes the necessity of outputting therapy, it can, as an alternative to the other methods listed, plan the output of therapy for a certain time of day, i.e., all subunits synchronously release their partial contributions at that time of day, or it can plan a programmed duration with a delay from a certain point in time. Such a time point in time can be, e.g., the synchronous command to start the charging process. The clocks are enabled to synchronize with external clocks (e.g., radio-controlled clocks). Alternatively, the clocks synchronize themselves only within the implanted system. The clocks are synchronized either in an event-driven manner at the point in time of detection or at regular intervals, to ensure accuracy within 5 ms.

In a preferred embodiment of the invention, a subunit detects physiological parameters that require therapy, and following that outputs a voltage directed at the therapy site. The at least one other subunit detects the voltage output of the first subunit, and following that also outputs voltage targeted at the therapy site.

The subunits have an electrode pole that is galvanically coupled to the tissue and that preferably is put on the housing of the subunit (housing pole). In a preferred embodiment, this electrode pole is the housing itself of the respective subunit, this housing then being realized so that it is electrically conductive (e.g., metal). This electrode pole can optionally be additionally connected for outputting therapy. This information can also be encoded by the therapy command coming from the subunit in the master role. This electrode pole (housing pole) is optionally also used for sensing.

Alternatively or additionally, the connection cable between the subunits has additional electrode poles on it for the sensing function, these additional electrode poles being connected with the sensing/detection units.

Signals from sensing electrodes, which are only connected with slave subunits, are processed by the sensing/detection unit of the respective slave subunit and the result is transferred to the master subunit through the communication unit. To accomplish this, the communication links between the subunits are bidirectional.

According to one embodiment of this invention, the inventive arrangement is a device for high-voltage therapy that is implanted in the body 120 or applied to the body, this device for high-voltage therapy consisting of at least two subunits that contain energy storage elements and that are connected with one another by at least one galvanic connection cable that is electrically insulated from the body. These subunits are electrically connected with one another in such a way that the therapeutic effect is achieved by synchronized superimposition of partial contributions from the individual subunits to the total electric power. The synchronization of the partial contributions is accomplished by galvanic, electromagnetic, optical, or mechanical/acoustic communication between the subunits.

The system for defibrillation of the heart should, on the one hand, be small and economical, and on the other hand it should be highly effective and very reliable. To accomplish this, it should be built from components and assemblies that have smaller requirements on dielectric strength than the voltage required in the area where the effect is applied (in the heart). This system should, e.g., be able to defibrillate successfully even from outside the chest (the ribs), although the components are designed for voltages under 1,000 V.

Other features which are considered as characteristic for the invention are set forth in the appended claims.

Although the invention is illustrated and described herein as embodied in a device for high-voltage therapy, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1A is an illustration showing an example of the inventive arrangement, which has two subunits and is arranged on or in the body of a patient;

FIG. 1B is an illustration showing an example of the inventive arrangement, in which the subunits are arranged in one housing;

FIG. 2A is an illustration showing an example of the interconnection of the inventive subunits;

FIG. 2B is an illustration showing an example of the interconnection of the inventive subunits corresponding to the embodiment in FIG. 1b

FIG. 3 is a graph showing examples of a superimposition of two voltages according to embodiments of this invention;

FIG. 4 is an illustration showing an example of the interconnection of the inventive subunits if arrangement has three subunits;

FIG. 5 is an illustration showing an example of a preferred geometric shape of the subunits; and

FIGS. 6A-6J are illustrations showing sample implantation arrangements of the inventive arrangement.

### DETAILED DESCRIPTION OF THE INVENTION

In the figures, all elements that are functionally the same or have the same effect are labeled with the same reference numbers. The figures are schematic representations of the invention. They depict non-specific parameters of the invention. Furthermore, the figures only reproduce typical embodiments of the invention, and are not intended to limit the invention to the embodiments shown.

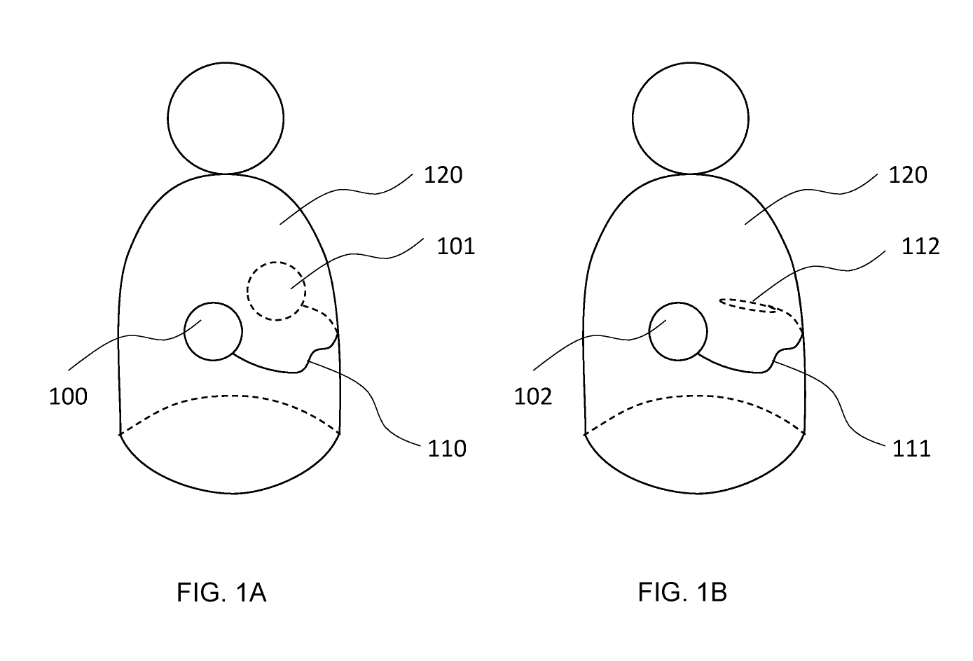

Referring now to the figures of the drawings in detail and first, particularly to FIG. 1A thereof, there is shown a system implanted in a body 120, the system consisting of subunits 100 and 101, which are connected with one another by means of a galvanic connection cable 110 (potential connector), which is electrically insulated from the body.

FIG. 1B shows an alternative implementation, in which the subunits 100 and 101 are encapsulated in common housing 102 and are connected, through a lead 111, with a remote counter electrode 112.

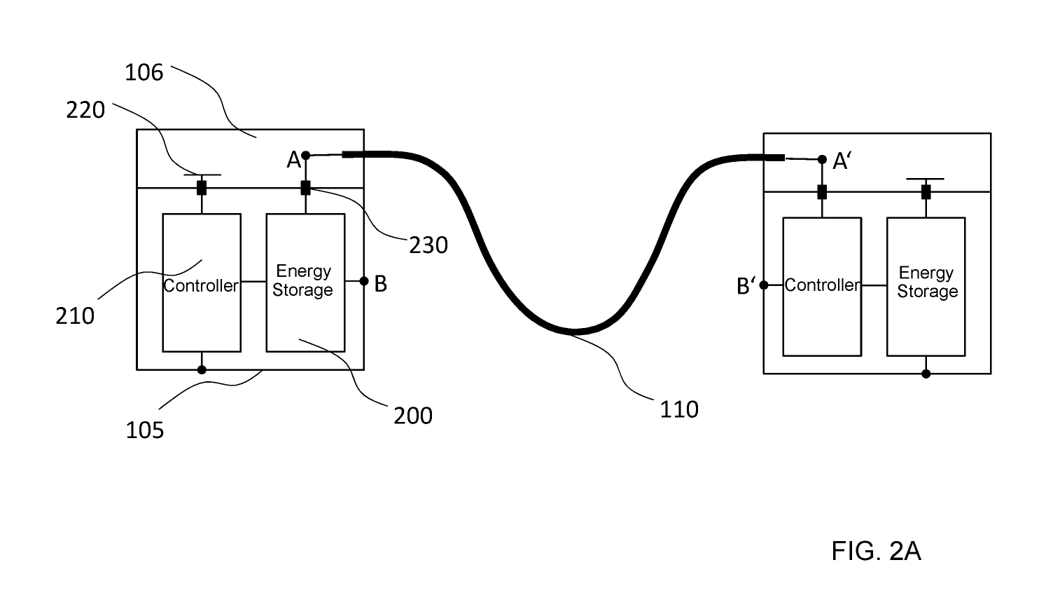

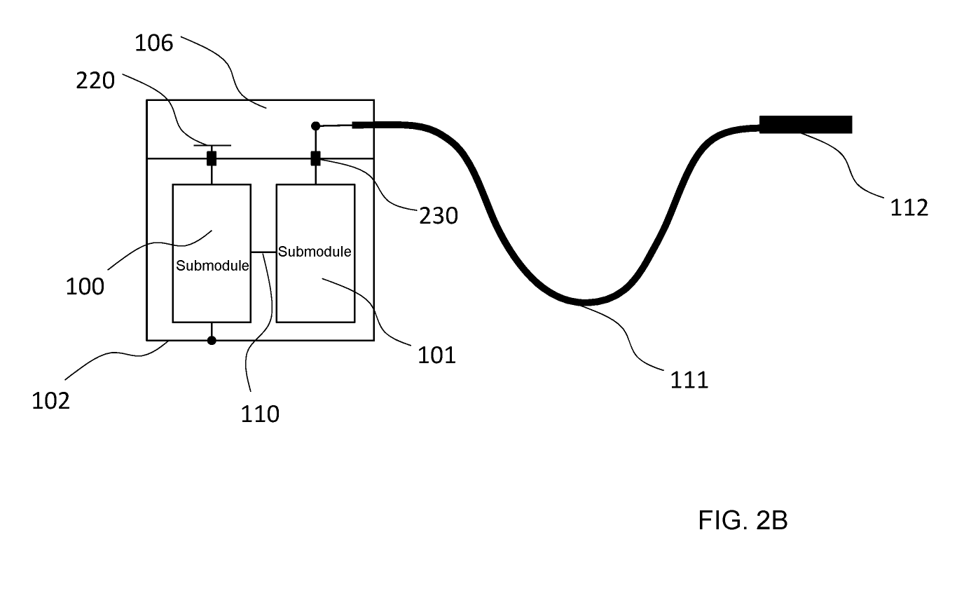

FIG. 2A shows the interconnection of the subunits 100 and 101 and the principle construction of the subunit 100 containing: a housing 105, which is preferably electrically conductive, and the terminal area (header) 106, the electrical connections passing from the inside of the housing 105 through hermetic feedthroughs 230 into the terminal area 106. Block 200 represents the combined energy storage elements. Reference number 210 identifies a controller and contains a communication unit that exchanges information with other subunits or external devices through a transceiver element 220. The latter is, e.g., an antenna for electromagnetic communication or a piezo element for communication in the ultrasonic range. Furthermore, the controller 210 contains a detection unit, which analyzes a voltage UAB to detect electrical cardiac activity (that is, it performs sensing).

FIG. 2B shows the alternative construction of the solution according to FIG. 1B.

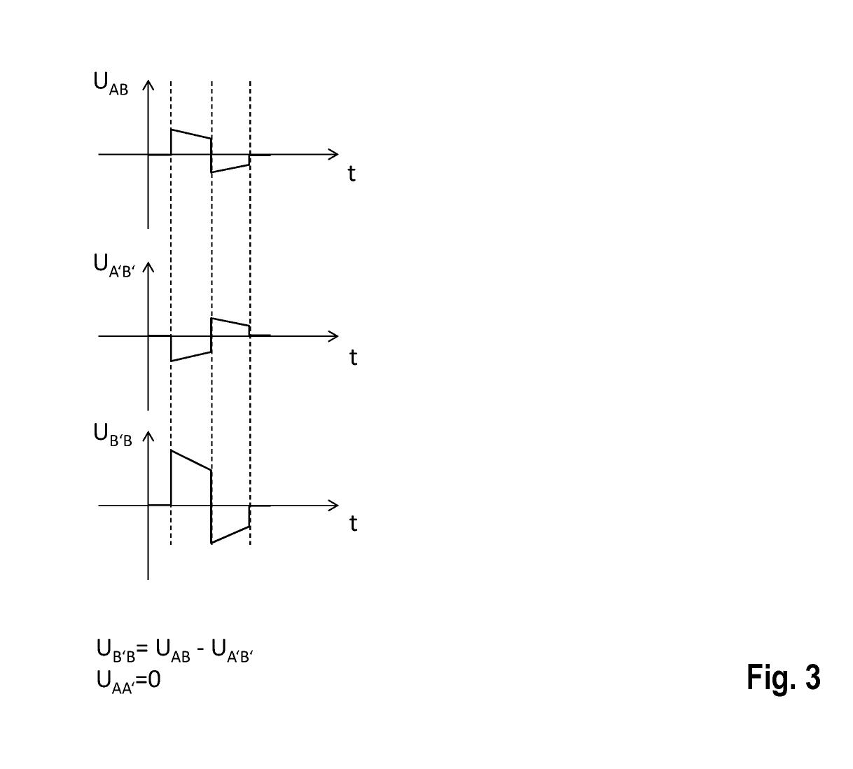

FIG. 3 illustrates the principle of the synchronized superimposition of partial contributions to obtain the total electric power from the individual subunits corresponding to FIG. 2. UAA′=0 is provided by the connection cable 110 (potential connector).

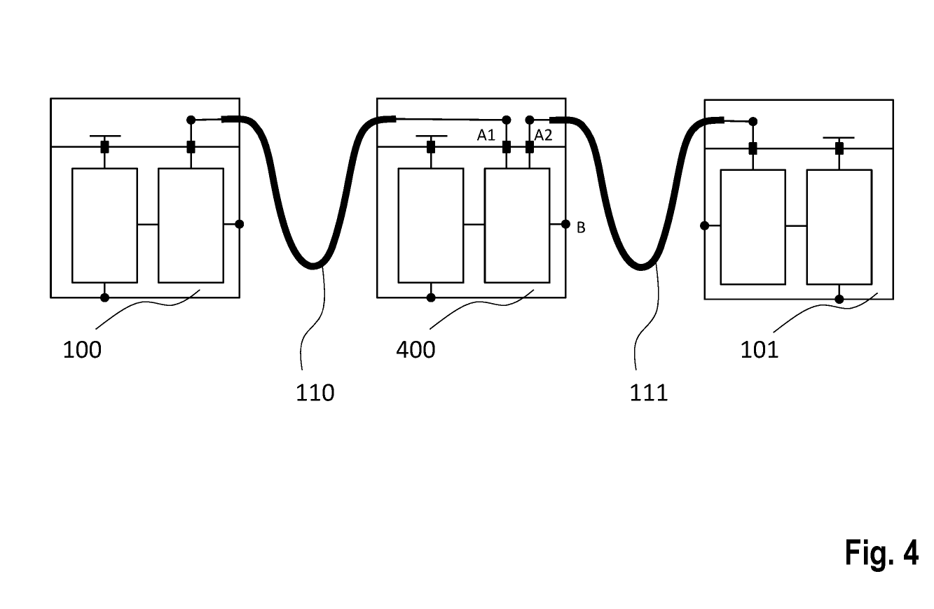

FIG. 4 shows a sample interconnection of 3 subunits (generally speaking there can be multiple subunits). This makes it possible to adjust various therapy vectors, depending on how the electrically conductive housing (or housing poles) are additionally connected through the controllers. In principle, a subunit 400 is built like the subunits 100 and 101, except that it can connect two (or more) connection cables 110 and 111.

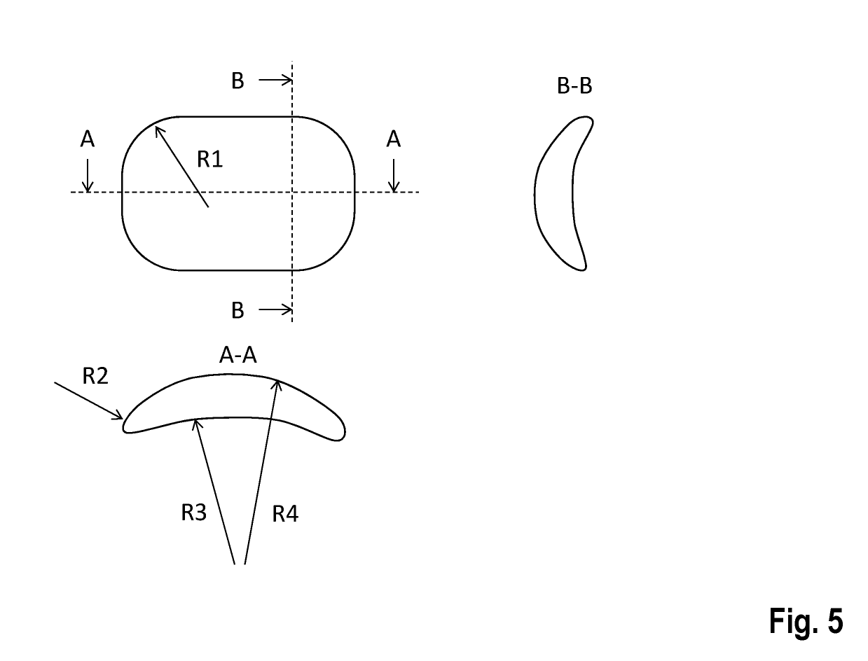

FIG. 5 shows a preferred geometric shape of the subunits.

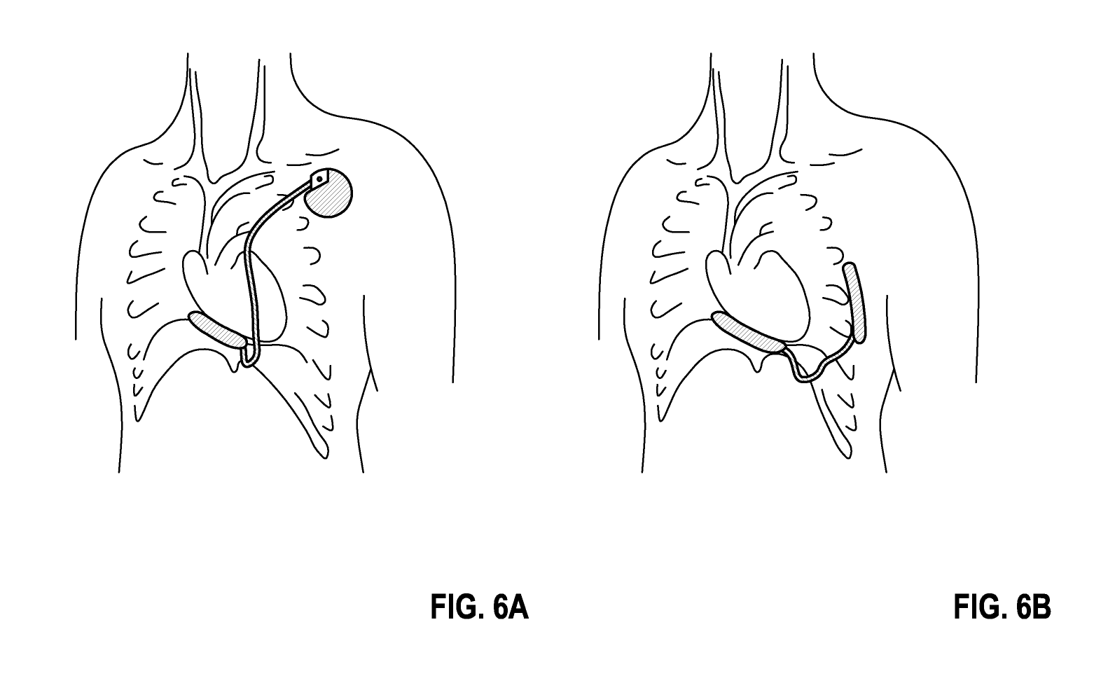

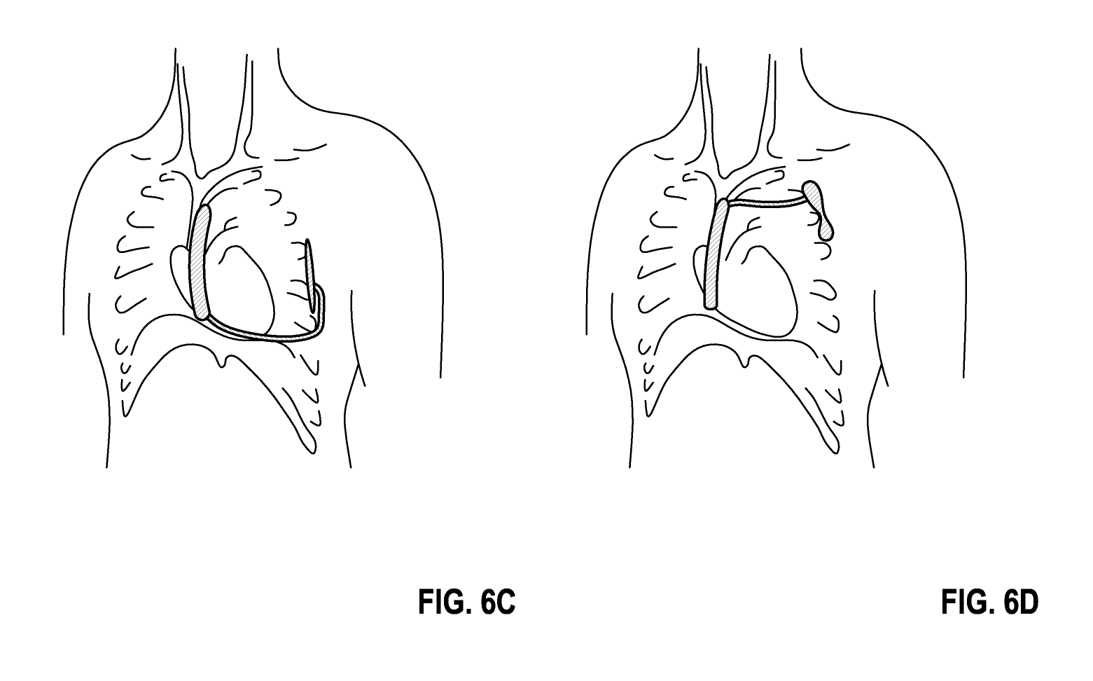

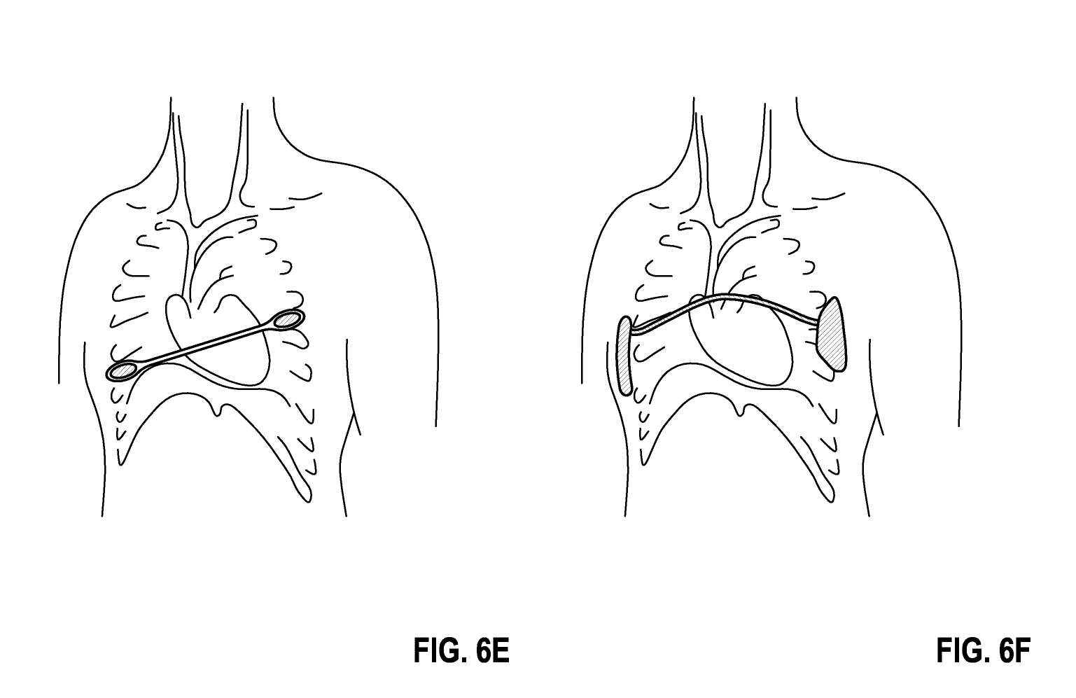

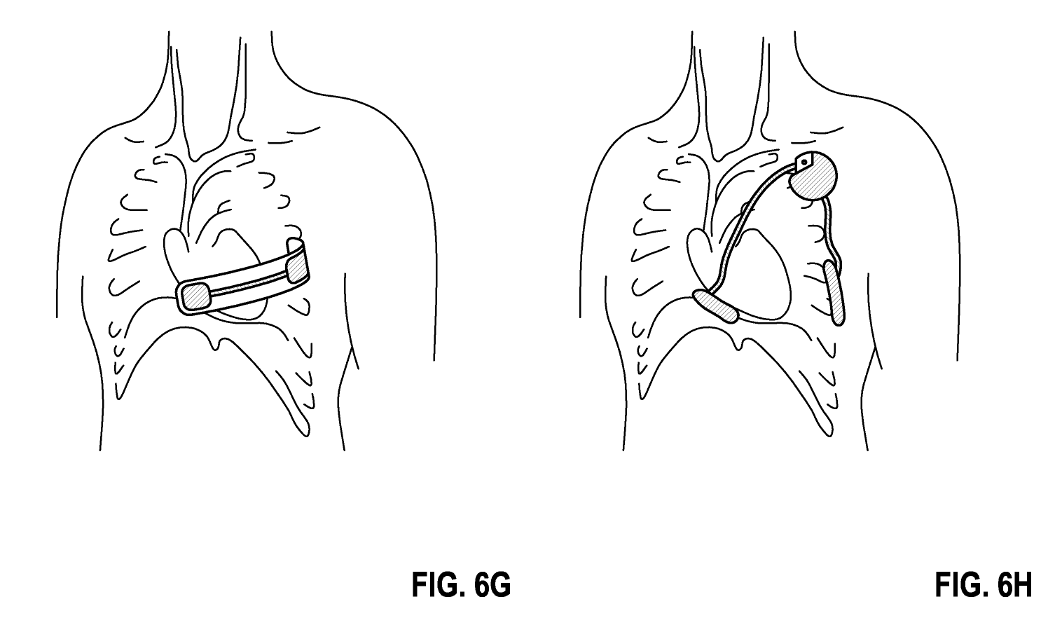

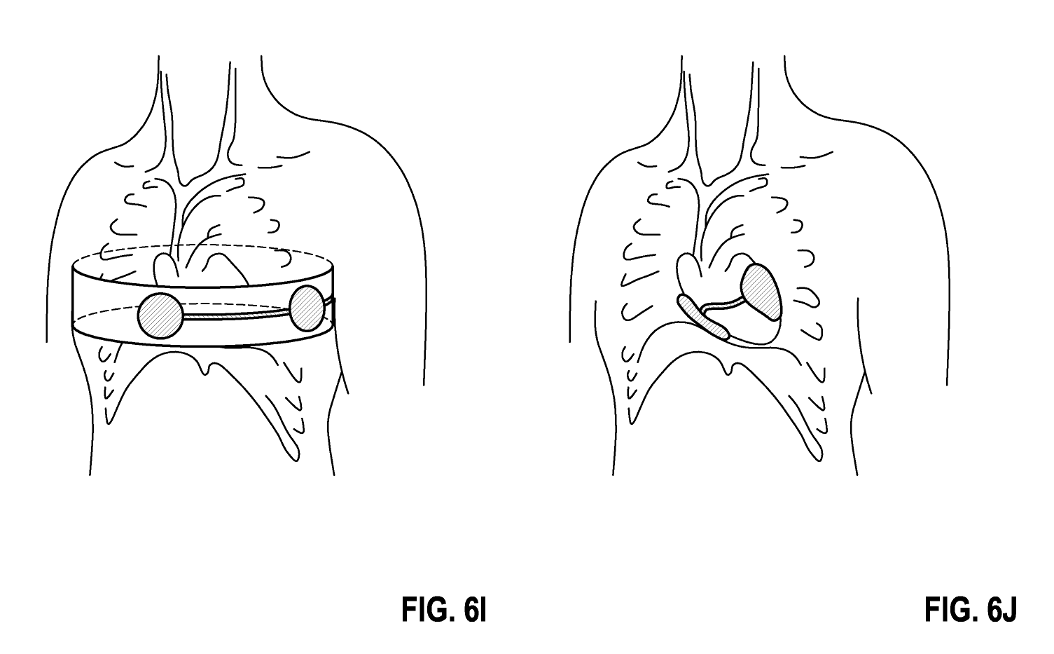

FIG. 6A through 6J show preferred implantation arrangements of the system.

The subunits have at least one connection, in order to connect it with at least one other subunit through a connection cables 110.

The energy storage elements are batteries (primary cells and/or secondary cells) and capacitors, or a combination of them; FIG. 2 represents them combined together in block 200. Furthermore, the block 200 contains energy conversion means, such as, e.g., a charging circuit that converts low battery voltages into high therapeutic voltages under the control of controller 210. The block 200 also contains a switch (therapy switch), which additionally connects the therapeutic voltage (partial amount) corresponding to the application, under the control of controller. Thus, in the example shown in FIG. 4, a corresponding switch position can cause this voltage to be applied between the contact points A1 and A2, A1 and B, or A2 and B.

The subunits are connected with one another and synchronized in such a way that the therapeutic voltage results by summation of the partial voltages generated by the individual subunits. FIG. 3 illustrates this in an example.

The subunits are connected together and synchronized in such a way that the therapeutic current results by summation of the partial currents generated by the individual subunits. For the addition of the currents (e.g., parallel connection of the subunit), the connection cable contains more than only one conductor.

One of the subunits is realized or programmed/configured as master, and the other(s) are realized or programmed/configured as slave(s)

At least the subunit working as master has a detection unit (including a sensing unit) that derives the necessity of outputting therapy from analyzing intracorporeal signals. The sensing unit and detection unit are part of the controller 210, so they also have access, through their connection to block 200, to the measuring voltage UAB (sensing signals).

One inventive embodiment provides that multiple subunits or all subunits perform detection, the first one that makes a detection decision assuming the master role. The subunit that has become master puts the other subunits into the slave role.

Each subunit contains one controller to trigger the therapy command.

Each of the subunits contains a communication unit, which serves to transmit and receive the therapy command, among other things. In this case, the communication information is the therapy command. Transmission and reception should be understood to include galvanic coupling.

The subunit working as master forwards the therapy command both to its own therapy unit (realized by the block 200) and also to the slave subunits

The therapy command encodes both the point in time at which the therapy is output, and also the polarity of and the duration for which the partial energies of the individual subunits are additionally connected. This ensures that the partial contributions of the individual subunits are superimposed synchronously and with the right sign (concerning this, see the example in FIG. 3). The synchronization is accurate to within 5 ms.

The subunits possess time measurement devices that are accurate to within 5 ms, i.e., clocks that know the absolute time and/or can measure time intervals. If the master recognizes the necessity of outputting therapy, it can, as an alternative to the other methods listed, plan the output of therapy for a certain time of day (all subunits synchronously release their partial contributions at that time of day) or it can plan a programmed duration with a delay from a certain point in time. Such a time point in time can be, e.g., the synchronous command to start the charging process. The clocks are enabled to synchronize with external clocks (e.g., radio-controlled clocks). Alternatively, the clocks synchronize themselves only within the implanted system. The clocks are synchronized either event-driven at the point in time of detection or at regular intervals, to ensure accuracy within 5 ms.

The subunits have a electrode pole that is galvanically coupled to the tissue and that preferably is put on the housing of the subunit (housing pole). In a preferred embodiment, this electrode pole is the housing itself of the respective subunit, this housing then being realized so that it is electrically conductive (e.g., metal).

This electrode pole can optionally be additionally connected for outputting therapy. The therapy command coming from the master subunit also encodes the information about whether it should be additionally connected.

This electrode pole (housing pole) is optionally also used for sensing.

Alternatively or additionally, the connection cable between the subunits has additional electrode poles on it for the sensing function, these additional electrode poles being connected with the sensing/detection units.

Signals from sensing electrodes that are only connected with slave subunits are processed by the sensing/detection unit of the respective slave subunit and the result is transferred to the master subunit through the communication unit. To accomplish this, the communication links between the subunits are bidirectional.

The master also receives feedback about the progress of the charging process to ensure that the command to output therapy (that is, providing synchronized partial amounts) is issued only once all subunits have completed the charging process.

The sensing/detection unit is protected by circuit breakers from overvoltages due to the output of therapy from its own subunit or from other subunits of the system. These switches are opened by the controller shortly before the therapy is output, and are reclosed after that. Optionally, the sensing/detection unit is additionally protected from overvoltages by passive protective measures (also in the case of external defibrillation). To accomplish this, one sample embodiment uses protective diodes.

In order that the electrodes involved in the therapy can also once again serve as sensing electrodes soon after therapy, the subunits possess devices to discharge the electrode after-potentials that are designed, e.g., as short circuit switches.

In one embodiment, the communication between the subunits is galvanic. This communication signal is preferably picked up and processed by the sensing unit. The invention provides the now described galvanic communication solutions.

The subunits have a bifilar/multifilar connection between them, i.e., the connection conductor also contains, in addition to the potential connector, at least one other lead. The electric circuit is closed through a pair of such connections. In a special embodiment, galvanic synchronization is accomplished using the pace and sense channels that are present in conventional ICDs, e.g., for atrium and ventricle.

The connection conductor contains only the potential connector. In this case, the electric circuit for communication is closed through the body. For this alternative, the invention provides the following implementations.

The electric circuit is fed an alternating current whose frequency and amplitude do not have a stimulating effect (either for muscles, nerves, or heart). This frequency is >1 kHz and its amplitude is <1 mA. Optionally, these values are individually programmable. The alternating current carries the communication information. The modulation is performed by a zero mean process (that is, no DC signals pass through the body). Preferred modulation methods are frequency modulation, pulse-width modulation, and possibly also amplitude modulation.

This electric circuit has pulses fed into it. The information is encoded by means of the pulse width and pulse interval. Preferably, the pulse width is less than 10 ms (especially preferably <2 ms), and the pulse interval is greater than 20 ms (especially preferably >80 ms).

One implementation option uses subthreshold pulses, which stimulate neither muscles, nerves, or heart

Another implementation option uses pulses that are super-threshold for myocardium. In this variant, the pulses are designed so that they also have therapeutic effect, in particular in the form of antitachycardia pacing (ATP). The subunits are synchronized to output the high-voltage therapy (to output their partial contributions to it) through a predetermined pattern (pulse widths and pulse intervals that are design-based or are programmed). While the synchronization is in progress, if the master unit decides to cancel therapy (that is, it does not close its therapy switch), the slave subunit is nevertheless triggered, however this partial amount does not reach the body, since the subunits are connected in series (the electric circuit in the master remains open).

A special embodiment provides that such a pulse is the therapy pulse itself. The master releases its partial amount, while the slave units still have their therapy switches open. The electric circuit first closes only by means of the sensing units of the slave. These sensing units of the slave pick up the therapy pulse of the master, and, if a programmable threshold amplitude is exceeded, also release their partial contributions with a reaction time of <5 ms.

In another embodiment, the communication between the subunits is electromagnetic. To accomplish this, the invention provides the now described solutions. Namely electromagnetic communication or radio frequency communication allows data and information exchange between the units 100, 101 to take place independently of the galvanic connections 110 and with a substantially higher speed and, as a result, shorter reaction times. The electromagnetic communication is carried out using the MICS band radio system that the units contain as standard equipment.

For energy reasons, the electromagnetic communication in both units is only activated in case of need:

a) It is activated every time there is a detection that leads to therapy or shock output (e.g., during the charging process).

b) A detection message of the units among one another through the bifilar connection 110. If the bifilar connection is provided by the ventricular or atrial pace/sense channel, a signal corresponding to the detection criterion is stimulated. The cases “all units detect” and “one unit detects” are covered, to guarantee that all involved units activate their electromagnetic communications system. The electromagnetic communication is activated periodically or by agreement, e.g., to determine the status or exchange data, to test the connection, etc.

c) The first detecting unit always functions as master to trigger therapy via electromagnetic communication.

In another embodiment, the subunits communicate among one another mechanically/acoustically, especially in the ultrasound range.

Another alternative provided by the invention for synchronizing the therapeutic partial contributions is to trigger the IEGM/ECG picked up by the therapy system. In a preferred implementation, interference is minimized by evaluating only limited frequency ranges and/or using morphological features of the IEGM/ECG.

The inventive implantable system is able to communicate with other implanted systems such as pacemaker systems (especially ILP), monitoring systems (e.g., loop recorders) and sensors (e.g., blood pressure sensors), and also with external devices (e.g., for programming and data transmission). The controller 210 also transmits therapy commands to pacemaker systems integrated into the therapeutic concept (e.g., post-shock pacing or ATP), or receives information from these devices for the purpose of expanded sensings (i.e., more reliable rhythm detection, discrimination of the origin of tachycardia, assessment of the hemodynamic relevance of dysrhythmia on the basis of pressure signals).

The subunits have a geometric shape that is adapted as well as possible to the physiological shape of the thorax (rib curvature), add little thickness, and minimize pressure points. A sample implementation is shown in FIG. 5. To accomplish this, the housing is preferably spoon-shaped and is dimensioned so that its length and/or width are at least 5 times greater than the thickness. The radii of the peripheral contour are at least R1>5 mm. The radii of the cross sectional contour are at least R1>1 mm. The radii of the spoon-shaped curvature are at least R4>30 mm. The concave curvature described by R3 is less than or no more than that described by R4. In a preferred embodiment, R3 is also <1,000 mm.

The inventive connection cable 110 between the subunits and the lead 111 are designed as follows:

a) The insulation is a biocompatible material, preferably silicone or polyurethane or a combination of them.
b) The insulation represents the lead body and has at least one lumen through which the potential connector is guided.
c) The potential connector consists of wires that are highly electrically conductive (preferably DFT® wire).
d) The potential connector is additionally insulated by material that slides well in the lumen.
e) The lead body has more than one lumen, every lumen guiding part of the wires of the potential connector (providing redundancy to increase reliability). These wires are brought together to the respective plug at the ends of the lead. The lumina in the lead body are guided to have a helicoidal twist, to minimize mechanical stresses when bent.
f) The lead body has supporting structures for reinforcement against squeezing stress. These structures are in the form of an interlayer concentric to the lead cross section, the potential connector running inside.
g) The materials of the supporting structures are themselves preferably biocompatible and are, e.g., metal fibers, aramid fibers, or silicones of higher Shore hardness.
h) The supporting structures are braided or are in the form of rings distributed over the length (that is, the structure is that of the trachea).

The inventive device is preferably implanted as shown in FIGS. 1A and 1B and in FIGS. 6A through 6J, and as is explained below.

FIG. 6A: Abdominal cavity.

Device 1 is located in the abdominal cavity. The cable is tunneled past the xiphoid process over the sternum to device 2, which is located beneath of the clavicle at the typical ICD site.

FIG. 6B: Abdominal cavity and on the side of the chest.

Device 1 is located in the abdominal cavity. A cable is subcutaneously guided next to the xiphoid process to device 2, which is arranged on the side of the chest.

FIG. 6C: Substernal arrangement.

Device 1 has a long stretched-out very thin shape and is placed beneath the sternum. The cable is either tunneled to device 2 through the intercostal space, as is shown in the picture, or it leaves the chest near the xiphoid process to get to device 2, which is seated on the left side of the chest.

FIG. 6D: Sternal arrangement.

Device 1 has a long stretched-out, very flat shape and is placed on the sternum. The cable is subcutaneously guided outside the chest to device 2 on the side.

FIG. 6E: Intercostal arrangement.

Devices 1 and 2 have a narrow shape and are connected with a cable that is subcutaneous, but outside the chest. Each is arranged in the intercostal space between a pair of ribs. It is also conceivable for one or both devices to be wider and to extend over multiple intercostal gaps.

FIG. 6F: Chest.

Both devices have a subcutaneous, possibly submuscular seat outside the chest. They are connected together by means of a cable that runs over the sternum.

FIG. 6G: Flat design with integrated application.

Devices 1 and 2 are very flat and ergonomically designed and are integrated into a flat band that is subcutaneously implanted on the chest.

FIG. 6H: Possible implementation with 3 devices.

Two devices are implanted subcutaneously, but outside the chest, and one is implanted in the abdominal cavity. Every device has the potential to shock. The shock vector can be variably adjusted. A useful arrangement can be to shock from the abdominal cavity parallel to the other two devices.

FIG. 6I: Chest belt.

In this implementation, the system consisting of at least 2 devices is integrated into a belt or a vest. This form of the implementation serves for temporary use of a cardioverter system.

FIG. 6J: Epicardial embodiment.

In this embodiment, a system consisting of 2 very thin, ergonomically pre-bent devices is set onto the epicardium.

The inventive solution makes it possible to realize an S-ICD system on the basis of conventional, economical implant technology. The functionality can be implemented by adapting the software, without having to develop and manufacture new hardware. The volume to be implanted is distributed, and adds less thickness than conventional S-ICDs (that is, it is more comfortable to wear).

1. A configuration for electrical therapy on a patient, the configuration configured to produce at least one electrical therapeutic voltage, which acts at a therapy site in or on a patient's body, the configuration comprising:
subunits including at least one first subunit and one second subunit, each of said subunits having at least one energy storage, an amount of energy storable by every individual said energy storage being smaller than an amount of energy required to produce the electrical therapeutic voltage for therapy.

2. The configuration according to claim 1, wherein each of said subunits having at least one of the following components:
a detection unit to detect body parameters;
a therapy unit to produce and/or output the electrical therapeutic voltage for electrical therapy;
a controller; and/or
a communication unit.

3. The configuration according to claim 1, wherein each of said at least one first and second subunits being configured to produce a voltage by means of energy from said energy storage, and the configuration being configured to produce the electrical therapeutic voltage by superimposing voltages produced by said at least one first and second subunits.

4. The configuration according to claim 1, further comprising:
a first outer housing, said at least one first subunit is disposed in said first outer housing; and
a second outer housing, said second subunit is disposed in said second outer housing.

5. The configuration according to claim 3, further comprising a common outer housing, said at least one first and second subunits are disposed in said common outer housing.

6. The configuration according to claim 3, wherein the voltages produced by said first and second subunits are superimposed so that an amplitude of a superimposed voltage is higher than an amplitude of the voltage produced by one of said subunits.

7. The configuration according to claim 1, wherein voltages produced by said first and second subunits are superimposed so that the voltages are essentially phase-synchronized at the therapy site.

8. The configuration according to claim 1, wherein each of said subunits is configured to communicate with at least one other of said subunits, a communication being galvanic, electromagnetic, optical, mechanical, and/or acoustic.

9. The configuration according to claim 2, wherein said controller is configured to issue a therapy command carrying information about at least one of the following parameters:
information about a phase of a voltage provided by a respective one of said subunits;
a point in time that the electrical therapy is output;
a polarity of the electrical therapy that is output; and/or
a duration of the electrical therapy that is output from said respective subunit.

10. The configuration according to claim 2, wherein one of said subunits assumes a master role with respect to producing and/or outputting a voltage, and at least one other of said subunits assumes a slave role, so that said at least one subunit playing the slave role orients itself about a point in time and/or polarity and/or duration of a therapy output on a basis of said subunit playing the master role.

11. The configuration according to claim 10, wherein said one subunit that assumes the master role first detects a body parameter calling for the electrical therapy, namely said one subunit detects the body parameter calling for the electrical therapy before all other said subunits.

12. The configuration according to claim 1, further comprising at least one connection configuration that electrically connects said subunits and is electrically insulated from the patient's body.

13. The configuration according to claim 12, wherein said subunits having a series, anti-series, parallel, or star-shaped connection through said connection configuration.

14. The configuration according to claim 12, wherein voltages produced by said subunits are generally phase-synchronous at the therapy site through said at least one connection configuration.

15. The configuration according to claim 1, wherein said subunits:
further comprising one battery each; and/or
further comprising one capacitor each; and/or
each representing a subcutaneously implantable cardioverter-defibrillator (sICD).
